(19) United States
(12) Patent Application Publication
(10) Pub. No.: US 2010/0198019 A1
(43) Pub. Date: Aug. 5, 2010

(54) SUTURE MANAGEMENT APPARATUS FOR SURGICAL PORTAL APPARATUS INCLUDING INTERLOCKING CAP

(75) Inventor: Richard Bergeron, Wallingford, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/652,381

(22) Filed: Jan. 5, 2010

Related U.S. Application Data
(60) Provisional application No. 61/148,470, filed on Jan. 30, 2009.

(51) Int. Cl. A61B 1/32; A61B 17/04

(52) U.S. Cl. 600/235; 606/148

(57) ABSTRACT

A surgical portal apparatus for use in a surgical procedure incorporating one or more sutures is provided. The surgical portal apparatus includes a portal member defining a longitudinal axis and having a longitudinal opening therethrough, and a suture management device operably connected to the portal member. The suture management device includes a first member and a second member mountable to the first member. The first member and the second member have cooperating surfaces adapted to selectively secure the at least one suture in a predetermined relation with respect to the portal member.

### CROSS REFERENCE TO RELATED APPLICATION

The present application claims the benefit of and priority to U.S. Provisional Application Serial No. 61/148,470 filed on Jan. 30, 2009, the entire contents of which are incorporated herein by reference.

### BACKGROUND

1. Technical Field

The present disclosure relates to a trocar and other surgical portal apparatus, and more particularly, relates to a suture management apparatus for surgical portal apparatus that includes an interlocking cap.

2. Background of Related Art

Trocars and other surgical portal apparatus are known, as are myriad procedures that may be preformed using such assemblies. Many of the minimally invasive procedures performed through access assemblies necessitate or are simplified by the use of one or more sutures passing through the surgical portal apparatus. Sutures extending into a body cavity through a surgical portal apparatus may be used to, for example, temporarily retain tissue, manipulate tissue, anchor tissue or operate peripheral devices. In an attempt to reduce the number of incision sites required to complete a given surgical procedure, a single surgical portal apparatus may be used to pass one or more sutures into a body cavity, in addition to providing access for one or more devices. A single anchor device may have numerous suture ends that extend therefrom and through the surgical portal apparatus. The sutures extending through the surgical portal apparatus may become tangled as each is manipulated or as one or more instruments are inserted and withdrawn from the assembly. Also, a surgeon may confuse the suture ends during the course of a surgery. Tangling or confusion of the suture ends may unnecessarily complicate the procedure and increase time necessary to complete the procedure.

Therefore, it would be beneficial to have an apparatus for use with a surgical portal apparatus for managing sutures.

### SUMMARY

A surgical portal apparatus for use in a surgical procedure incorporating one or more sutures includes a portal member defining a longitudinal axis and having a longitudinal opening therethrough and a suture management device operably connected to the portal member. The suture management device includes a first member and a second member mountable to the first member. The first member and the second member have cooperating surfaces adapted to selectively secure the at least one suture in a predetermined relation with respect to the portal member. The first member may include at least one suture retaining slot. The second member includes at least one depending rib dimensioned for at least partial reception within the at least one slot of the first member. The at least one slot and the at least one rib may extend radially outwardly relative to the longitudinal axis.

In one embodiment, the first member may include a plurality of slots and the second member may include a plurality of ribs. The ribs are dimensioned and arranged to be partially received within respective slots to thereby releasably secure a plurality of sutures. The respective slots and recesses of the first and second members may be arranged in predetermined radial spaced relation.

The first member and the second member each define a longitudinal opening sized to receive a surgical object. The portal member may include a housing segment and an elongated segment extending from the housing segment. The suture management device is adapted to releasably engage the housing segment. Alternatively, the suture management device is integrally formed with the housing segment.

In another aspect, a suture management device includes an insert member configured to be operably connected to a portal member. The insert member includes a plurality of radially outward extending slots. A cap member includes a plurality of radially outward extending ribs configured to engage in the slots of the insert member, wherein engagement of the ribs in the slots securely retains at least one suture therein.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective side view of a surgical portal apparatus including a suture management apparatus according to an embodiment of the present disclosure;

FIG. 2 is a perspective side view of the cap member of the suture management apparatus of FIG. 1;

FIG. 3 is a perspective side view of the insert member of the suture management apparatus of FIG. 1; and

FIG. 4 is a perspective side view of a surgical portal apparatus including a suture management system according to another embodiment of the present disclosure;

### DETAILED DESCRIPTION

The surgical portal apparatus herein disclosed may be configured for use in various surgical procedures, including laparoscopic, endoscopic, arthroscopic and orthopedic surgery. The access assembly provides passage between a subject's body cavity and the outside atmosphere and is capable of receiving surgical instruments of various sizes and configurations. An embodiment of the presently disclosed access assembly is configured to receive, for example, clip appliers, graspers, dissectors, retractors, staplers, laser probes, photographic devices, endoscopes and laparoscopes, tubes, and the like. Such instruments are collectively referred to herein as “instruments” or “instrumentation.”

In addition to the instruments, the access assembly also allows the passage of one or more sutures therethrough, e.g., during an arthroscopic procedure. When several sutures are introduced into the subject's body through the access assembly, the sutures might tangle with each other or be confused by a surgeon. Suture tangle and/or confusion may, at the very least, inconvenience the clinicians conducting the surgical procedure. To minimize the possibility of sutures tangling with one another or a surgeon from confusing the sutures, the access assembly incorporates a suture retaining member for holding the one or more suture in place. The suture retaining member may also be used to maintain the one or more suture in a taut condition.

Referring now to the drawings wherein like reference numerals illustrate similar components throughout the several views, there is illustrated a suture management system in accordance with the principles of the present disclosure. In the following description, the term “proximal” refers to the portion of the access assembly that is closest to the clinician, whereas the term “distal” refers to the portion of the access assembly that is farthest from the clinician. As used herein, the term “subject” refers to a human patient or other animal. The term “clinician” refers to a physician, nurse or other care provider and may include support personnel.

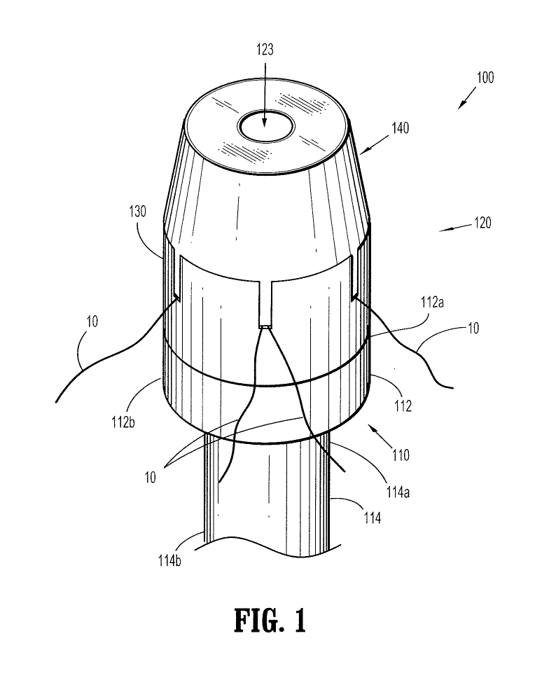

Referring initially to FIG. 1, a suture management system according the present disclosure is shown as suture management system 100. Suture management system 100 includes a surgical portal apparatus 110 and a suture management device 120.

Surgical portal apparatus 110 includes a portal member 112 and a sleeve 114 extending distally from portal member 112. Although the following discussion of suture management device 120 will be with respect to surgical portal apparatus 110, the aspects of the present disclosure should not be read as limited to the embodiments herein disclosed. Suture management device 110 may be modified for use with any surgical portal apparatus.

Still referring to FIG. 1, portal member 112 defines a substantially cylindrical housing having an open proximal end 112a and a substantially open distal end 112b. Open proximal end 112a is configured to engage insert 120. Distal end 112b of portal member 112 may be integrally formed with sleeve 114. Alternatively, portal member 112 may be configured for selectable engagement with sleeve 114. Portal member 112 may be construction of plastic, polymer or other like material. Portal member 112 may be disposable, or in the alternative, reusable. Portal member 112 may be rigid, or alternatively, substantially flexible. Portal member 112 may include one or more seal members (not shown) having any seal arrangement for receiving a surgical object in a sealing manner. Portal member 112 may further include one or more anchors (not shown) or other suture securing means for securing one or more suture “S” extending through surgical portal apparatus 110. Portal member 112 may further include an insufflation valve or port (not shown) configured to fill the body cavity of a patient with insufflation gas, saline or other suitable fluid.

Sleeve 114 is configured to be inserted through the skin into a body cavity with the aid of an obturator (not shown), or may instead, include a blade or piercing tip for penetrating through the skin and into a body cavity. Sleeve 114 forms a substantially tubular member having proximal and distal ends 114a, 114b and defining a first longitudinal passage 113 extending therebetween. Sleeve 114 may be composed of plastic, metal, polymers or the like. Sleeve 114 may be disposable, or in the alternative, reusable. Sleeve 114 may be rigid, or alternatively, sleeve 114 may be flexible. Sleeve 114 may be open, or instead, may be configured to include one or more seal members (not shown) having any seal arrangement along the length thereof.

With reference now to FIGS. 1-3, suture management device 120 includes a substantially cylindrical assembly configured to be received on proximal or housing end 112a of portal member 112. Suture management device 120 defines a longitudinal passage 123 configured for receipt of endoscopic, laparoscopic or other elongated instrument of various diameters. Suture management apparatus 120 includes an insert or first member 130 and a cap or second member 140 configured to engage insert member 130. Although suture management apparatus 120 is preferably used with a surgical portal apparatus having one or more seal members for receiving an instrument in a sealed fashion, it is envisioned that suture management apparatus 120 may include one or more seal members (not shown).

Referring now to FIG. 3, insert member 130 defines a substantially cylindrical body 132 having proximal and distal ends 132a, 132b and defining a longitudinal passage 133 extending therebetween. Distal end 132b includes a flanged or recessed portion 134 configured to be received in proximal end 112a of portal member 112. As shown, insert member 130 is configured to frictionally engage proximal end 112a of portal member 112; however, it is envisioned that insert member 130 and portal member 112 may be mechanically coupled, i.e. threaded engagement. Although shown as being received within proximal end 112a of portal member 112, it is further envisioned that insert member 130 may be configured to be received about portal member 112. Proximal end 132a of insert member 130 includes a plurality of radial extending slots 136. Slots 136 are spaced about proximal end 132a and extend outwardly from longitudinal passage 133. Insert member 130 may include any number of slots 136. Slots 136 may be of varying widths. Each of slots 136 is configured to receive at least one suture 10 (FIG. 1). As shown, each of slots 136 may include markings 138 to assist in identifying sutures 10 received therein. Markings 138 may be letters, numbers, symbols, colors or other identifying feature. As will be discussed in further detail below, markings 138 may also assist in aligning cap member 140 with insert member 130.

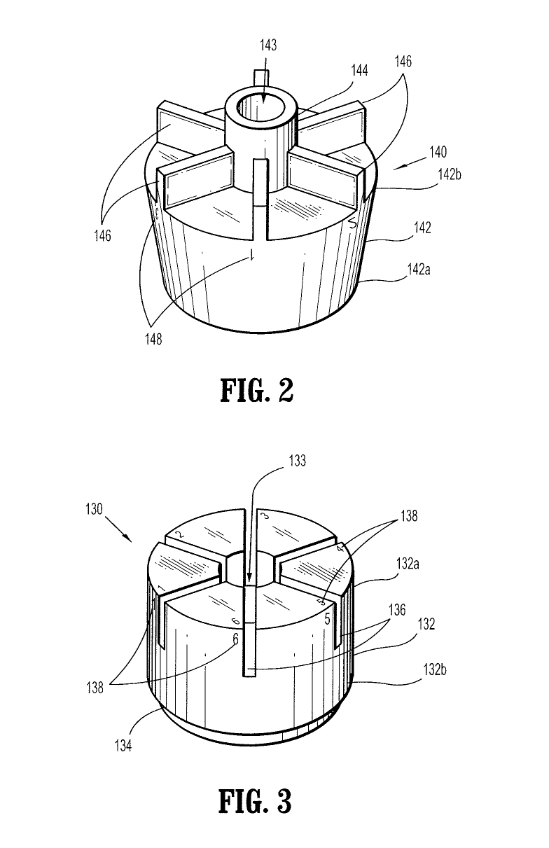

With reference now to FIG. 2, cap member 140 defines a substantially frustro-conical body 142 having proximal and distal ends 142a, 142b and defining a longitudinal passage 143 therebetween. Distal end 142 of cap member 140 includes a distal extension 144 extending therefrom. Distal extension 144 defines a distal end of longitudinal passage 143 and is configured to be received within longitudinal passage 133 of insert member 130. Cap member 140 further includes a plurality of ribs 146 extending radially outward from distal extension 144. Ribs 146 correspond in number and placement to slots 136 formed in proximal end 132a of insert member 130. Each of ribs 146 is configured to be received within a corresponding slot 136 of insert member 130. Ribs 146 may be over-sized, include a coating (not shown), or otherwise be configured to frictionally engage slot 136 and/or sutures 10. Distal end 142b of cap member 140 may also include markings 148 corresponding to markings 138 on insert member 130. Marking 148 may assist in aligning ribs 146 with slots 136 and/or, in lieu of markings 138 on insert member 130, to identify sutures 10 within slots 136.

Depending on the configuration of surgical portal apparatus 110 and/or the preference of the user, insert member 130 of suture management apparatus 120 may be attached to surgical portal apparatus 110 during manufacture, by a surgeon prior to insertion of surgical portal apparatus 110 into a body cavity or following insertion of the surgical portal apparatus 110 into the body cavity. Cap member 140 may be provided separate from insert member 130, or alternatively, cap member 140 may be engaged with insert member 130. As discussed above, sleeve 114 of surgical portal apparatus 110 may be of the self-piercing type or may instead be inserted with the assistance of an obturator (not shown). Prior to receipt of sutures 10 (FIG. 1) within slots 136 of insert member 130, surgical portal apparatus 110 functions as a traditional access assembly. A seal member (not shown) disposed within portal member 112, sleeve 114 and/or suture management apparatus 120 may operate to receive instrument “I” in a sealed manner.

In operation, cap member 140 of suture management apparatus 120 is disengaged from insert member 130 in order to permit one or more sutures 10 to be received within slots 136 of insert member 130. The sutures 10 may be used to manipulate tissue or in conjunction with, e.g. an orthopedic procedure to connect tissue. The sutures 10 may extend from the operative site through sleeve 114 of portal apparatus 110. Once one or more sutures 10 are received within slots 136, cap member 140 is engaged with insert member 130 such that ribs 146 engage slots 136 formed in insert member 130. Sutures 10 are thus frictionally retained within slot 146 by ribs 136. Alternatively, or additionally, distal extension 144 of cap member 140 frictionally engages sutures 10 against an inner surface of insert member 130. Cap member 140 may be disengaged and reengaged from insert member 130 as necessary to add, remove and/or relocate sutures 10 within slots 136. In using suture management system 100 during a surgical procedure, sutures 10 that would otherwise become tangled, misplaced and/or confused, are instead securely retained by suture management device 120 and maintained separate from instrument “I”.

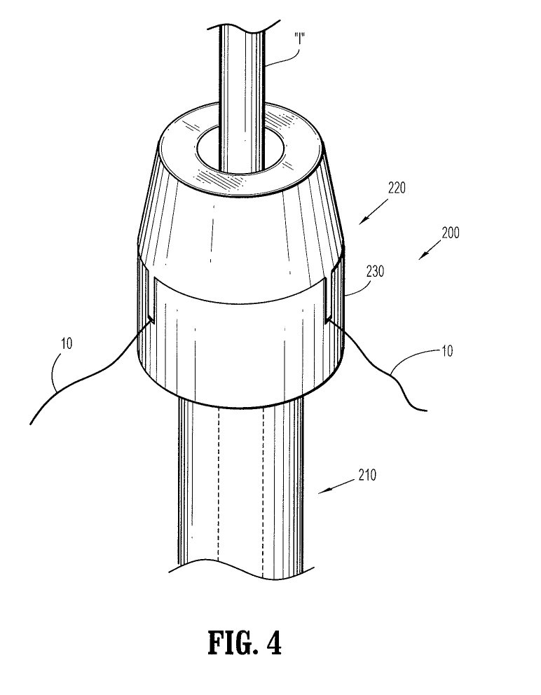

Turning now to FIG. 4, an alternate embodiment of a suture management system is shown generally as suture management system 200. Suture management system 200 is substantially similar in form and function to suture management system 100 described hereinabove. Suture management device 220 includes a suture management apparatus 220 and a surgical portal apparatus 210. Suture management apparatus 220, and insert member 230 in particular, is integrally formed with surgical portal apparatus 210. In this manner, insert member 230 and surgical portal apparatus 210 cannot accidentally become separated during a procedure.

Although the illustrative embodiments of the present disclosure have been described herein with reference to the accompanying drawings, it is to be understood that the disclosure is not limited to those precise embodiments, and that various other changes and modifications may be effected therein by one skilled in the art without departing from the scope or spirit of the disclosure.

What is claimed is:

1. A surgical portal apparatus for use in a surgical procedure incorporating one or more sutures, which comprises:
a portal member defining a longitudinal axis and having a longitudinal opening therethrough; and
a suture management device operably connected to the portal member, the suture management device including a first member and a second member mountable to the first member, the first member and the second member having cooperating surfaces adapted to selectively secure the at least one suture in a predetermined relation with respect to the portal member.

2. The surgical portal apparatus of claim 1, wherein the first member includes at least one suture retaining slot.

3. The surgical portal apparatus of claim 2, wherein the second member includes at least one depending rib dimensioned for at least partial reception within the at least one slot of the first member.

4. The surgical portal apparatus according to claim 3, wherein the at least one slot and the at least one rib extend radially outwardly relative to the longitudinal axis.

5. The surgical portal apparatus according to claim 4, wherein the first member includes a plurality of slots and the second member includes a plurality of ribs, the ribs dimensioned and arranged to be partially received within respective slots to thereby releasably secure a plurality of sutures.

6. The surgical portal apparatus of claim 5, wherein the respective slots and recesses of the first and second members are arranged in predetermined radial spaced relation.

7. The surgical portal apparatus of claim 6, wherein the first member and the second member each define a longitudinal opening sized to receive a surgical object.

8. The surgical portal apparatus of claim 1, wherein the portal member includes a housing segment and an elongated segment extending from the housing segment, the suture management device being adapted to engage the housing segment.

9. The surgical portal apparatus of claim 1, wherein the suture management device is integrally formed with the portal member segment.

10. The surgical portal apparatus of claim 1, wherein at least one of the first member and the second member include markings for identifying the one or more sutures retained therein.

11. The surgical portal apparatus of claim 1, wherein at least one rib of the second member is dimensioned for reception within the at least one slot of the first member in frictional relation therewith.

12. A suture management device for use with a portal member, the device comprising:
an insert member configured to be operably connected to a portal member, the insert member including a plurality of radially outward extending slots; and
a cap member including a plurality of radially outward extending ribs configured to engage in the slots of the insert member, wherein engagement of the ribs in the slots securely retains at least one suture therein.
